(19) United States
(12) Patent Application Publication
(10) Pub. No.: US 2010/0081694 A1
(43) Pub. Date: Apr. 1, 2010

(54) COMPOSITION COMPRISING AT LEAST ONE PPAR AGONIST AND A LIPID COMPONENT

(75) Inventors: Jan Kopecky, Prague (CZ); Martin Rossmeisl, Prague (CZ); Bjorn Refsum, Alesund (NO)

Correspondence Address: VOLPE AND KOENIG, P.C., UNITED PLAZA, SUITE 1600, 30 SOUTH 17TH STREET, PHILADELPHIA, PA, 19103, US

(73) Assignee: EPAX AS, Alesund (NO)

(21) Appl. No.: 12/566,751

(22) Filed: Sep. 25, 2009

Related U.S. Application Data
(60) Provisional application No. 61/101,178, filed on Sep. 30, 2008.

(51) Int. Cl. A61K 31/4439; A61P 7/00; A61P 3/00

(52) U.S. Cl. 514/342

(57) ABSTRACT

The present invention relates to compositions comprising at least one peroxisome proliferator-activated receptor (PPAR) agonist and a lipid component and to pharmaceutical ingredients comprising said ingredients. Such compositions are particularly suitable for the prophylaxis and/or the treatment of diseases such as diabetes, obesity and hypertriglyceridemia.

### FIELD OF THE INVENTION

The present invention relates to compositions comprising at least one peroxisome proliferator-activated receptor (PPAR) agonist and a lipid component and to pharmaceutical compositions comprising said ingredients. Such compositions are particularly suitable for the prophylaxis and/or the treatment of diseases such as diabetes, obesity and hypertriglyceridemia.

### BACKGROUND OF THE INVENTION

For maintenance of homeostasis, it is critical to keep a constant supply of glucose to the cells. Insufficient supply of glucose to the cells, as seen e.g. in insulin resistant states, would lead to numerous metabolic diseases, including obesity, heart disease, hypertension, diabetes, hypertriglyceridemia, chronic fatigue, accelerated aging, as well as many mental and emotional problems. For this reason, it is important to identify and, at the same time, treat this condition early before costly and disabling degenerative conditions arise that can ruin the quality of life as well as shorten it.

Diabetes or diabetes mellitus is a disease that occurs when the body cannot make use of the glucose in the blood for energy because either the pancreas is not able to make enough insulin or the insulin that is available is not effective, a situation called insulin resistance. There are two main types of diabetes mellitus: insulin-dependent (type 1) and noninsulin-dependent (type 2 or adult onset diabetes).

A third type of diabetes is the gestational diabetes that develops only in pregnant women with no previous history of diabetes. Nearly 135.000 U.S. women develop gestational diabetes each year. Typically, gestational diabetes clears up on its own after women have delivered their babies. But studies show that about 40% of women with gestational diabetes go on to develop type 2 diabetes within 15 years (NUI Publication No. 02-3873, May 2002).

In insulin-dependent diabetes (IDDM; type I diabetes), the pancreas makes little or no insulin because the insulin-producing beta cells have been destroyed. This might appear at any age but usually occurs between infancy and the late 30's, most typically in childhood or adolescence. Treatment consists of daily insulin injections or use of an insulin pump, a planned diet and regular exercise, and daily self-monitoring of blood glucose. If the level of insulin is too low for a long period of time, the body begins to break down its stores of fat for energy. This causes the body to release fatty acids which are then converted into ketone bodies or ketoacids that are toxic at high levels. The result is called ketoacidosis, a severe condition that may put a person into a coma if not treated right away.

In noninsulin-dependent diabetes (NIDDM), type 2 diabetes or adult onset diabetes (AOD), the body tissues become resistant to insulin. The patient's blood sugar level often rises gradually, taking several years to reach unsafe levels and cause symptoms. Thus, in some people, where the diabetic condition has not yet developed, normal or excessive levels of insulin compensate for such resistance. Over time however, insulin production often drops and resistance worsens. About 90-95% of all diabetic people have type 2 diabetes. It is more common in people over the age of 40.

The mechanisms behind the development of type 2 diabetes is not completely understood but it seems that when the lipid depots in our bodies are in the process of being overfilled, there is a spill-over of fatty acids in the circulation with a build-up of fatty components in the liver and the muscles which result in poor insulin effect in the muscles and liver but also in the fat tissue itself, a pathophysiological situation called insulin resistance. In spite of normal insulin production the normal insulin response in muscles and fat cells are reduced implying that more insulin has to be released in order to overcome the lack of effect.

Insulin mediates its actions through binding to insulin receptors which are membrane proteins integrated in the phospholipid bilayer membranes of cells. The receptor consists of a heterotetramer containing 2 α and 2 β glycoprotein subunits linked by disulphide bonds (Wilcox G. Insulin and insulin resistance. Clin Biochem Rev 2005; 26:19-39). The binding affinity and kinetics of the hormone is dependent on the consistency and fatty acid makeup of the phospholipid membrane of the cells.

Phospholipids consist of a hydrophilic, polar part and a hydrophobic non-polar part consisting of two fatty acids. There are two types of phospholipids, the glycerophospholipids and the sphingophospholipids given names after the glycerol or sphingosin backbone of the molecule. Sphingomyosin is the most common phospholipid of muscle cells, also called ceramide. The ionic part of the phospholipid molecule is relatively constant while the fatty acid makeup are subjected to dynamic changes (so called phospholipid remodelling) due to changes of fatty components in the diet.

The biological properties of the phospholipids in the cell membranes may change dependent upon the availability of the responsible phospholipid, fractions of the molecule when fatty acids are removed by actions of phospholipases or by the individual fatty acids. In sn-1 position there are usually saturated or mono-unsaturated fatty acids while in sn-2 position there are usually polyunsaturated fatty acids.

The absorption kinetics of phospholipids is poorly understood. Being part of the diet the phospholipids undergo partial degradation by phospholipases mainly produced in the exocrine pancreas. Phospholipase A1 and A2 remove the fatty acids from the sn-1 and sn-2 position, respectively, leaving the rest-molecule called lysophospholipids. The different components of the phospholipid mother molecule form spontaneously and enter the enterocytes of the small intestines while the fatty acids form micelles together with bile acids. It is not clearly established whether increased dietary intake can affect the interaction of phospholipids in the cell wall or if cellular needs for new-synthesis of phospholipids are the major determinant for the rate of synthesis.

The main cause of death in people with type 2 diabetes, regardless of sex or age, is heart disease. Other complications associated with diabetes include nerve damage (neuropathy) and vascular abnormalities in both small and large blood vessels. Heart attacks account for 60% and stroke for 25% of deaths in all diabetics. People with diabetes are at risk for heart-risk conditions that include hypertension, high triglyceride levels and lower high density lipoprotein (HDL), blood clotting problems, neuropathy, and silent ischemia.

Drug therapy is one common approach to treatment of adult onset diabetes. Oral agents such as sulfonylureas (e.g., glyburide, glipizide, glimepiride), meglitinides, biguanides, thiazolinediones, and alpha-glucosidase inhibitors, singly or combined, with or without insulin replacement therapy are used currently.

Metabolic Syndrome (MS) is a condition that promotes atherosclerosis and increases the risk of cardiovascular events through the collection of independent and related complications or disorders. The related complications or disorders of MS include dyslipidemia (hypertriglyceridemia and low HDL-cholesterol), a prothrombotic state, type 2 diabetes (adult onset diabetes), insulin resistance/hyperinsulinemia, and hypertension (Grundy, S. M. Am J. Cardiol. 81: 18B-25B, 1998).

Although the patient may not have any symptoms from MS, the attending physician could identify the following as signs of the condition: (1) elevated insulin levels, due to insulin resistance; (2) type 2 diabetes; (3) central obesity (a disproportionate amount of body fat in the abdominal region); (4) hyperlipidemia [high levels of fats (lipids) in the blood, in particular triglycerides]. In addition, the size of the LDLs may be smaller than usual, which is more likely to promote atherosclerosis; (5) low level of HDL cholesterol; (6) hypertension (high blood pressure); (7) elevated levels of blood factors that promote blood clotting, such as plasminogen activator inhibitor-1 (PAI-1) and fibrinogen; (8) hyperuricemia (high levels of uric acid in the blood); and (9) microalbuminuria (small amounts of the protein albumin, found on urine tests).

Currently, the only known treatment strategy that address all the factor of MS is weight loss and exercise. Medications are given but physicians would usually encourage the MS patients to change their life style such as decreasing the amount of fats and oils in their diet.

Besides the above-mentioned strategy, several groups have disclosed the use of specific drugs to treat MS and its related complications. Below is a brief summary of their disclosures.

U.S. Pat. No. 6,166,049 discloses a method for the treatment or prophylaxis of MS by administering an effective, non-toxic and pharmaceutically effective amount of an peroxisome proliferator-activator receptor-γ and -α (PPAR-γ and PPAR-α) agonist.

EP1585499 relates to a method of treating insulin resistance, adult onset diabetes and metabolic syndrome in a mammal by intravenously administering a therapeutically effective amount of a liposomal suspension of lipoprotein small unilamellar vesicles comprising predominantly phospholipids.

WO2006/062932 relates to a method utilizing a PPAR agonist and/or antagonist and omega-3 fatty acids for the treatment of subjects with hypertriglyceridemia, hypercholesteremia, mixed dyslipidemia, vascular disease, atherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes. Particularly, a synergistic result was obtained when Omacor® and fenofibrate were administered together.

WO2004/028469 disclose methods of treating patients with metabolic syndrome, prediabetes and/or type II diabetes mellitus by administering docosahexaenoic acid alone or in combination with diabetes related medications.

WO2007/053622 disclose that PPAR agonists, specifically PPARγ and PPARα are used as insulin sensitizers and regulators of lipid homeostasis in the treatment of diabetes. Thiazolidinediones, and particularly pioglitazone, is said to be agonists of PPARγ.

WO2005/037848 relates to a glycerophospholipid preparation useful in the treatment of various cognitive and mental conditions and disorders and for maintenance of normal functions of brain-related systems and processes.

Although a number of compositions have proven to be effective in reducing insulin resistance, type 2 diabetes and MS and its related complications, there is a continuing need to provide novel compositions having improved effect on the above-mentioned conditions and/or less side effects.

Surprisingly it has now been found that a composition comprising a lipid component rich in phospholipids is suitable for the prophylaxis and/or the treatment of the above-mentioned conditions. Further, the inventors of the present invention have also discovered that if said phospholipid composition is combined with a PPAR agonist, the biological effect is improved compared with each of the components separately. Said combination product allows for reduced dosages of each active ingredient to provide an effective pharmaceutical treatment and minimize unwanted side effects.

### SUMMARY OF THE INVENTION

A first aspect of the present invention relates to a composition comprising at least one peroxisome proliferator-activated receptor (PPAR) agonist and a lipid component, wherein at least 30% (w/w) of said lipid component are phospholipids.

A second aspect of the present invention relates to the composition according to the first aspect of the present invention for use as a medicament.

A third aspect of the present invention relates to a pharmaceutical composition comprising a pharmaceutically effective amount of the composition according to the first aspect of the present invention and a pharmaceutically acceptable carrier.

A fourth aspect of the present invention relates to use of the composition according to the first aspect of the present invention, for the preparation of a medicament for the prophylaxis and/or the treatment of a disease in a mammal, said disease being characterized by obesity and/or insulin resistance and/or glucose intolerance and/or hyperglycemia.

A fifth aspect of the present invention relates to use of the lipid component as defined in the first aspect of the present invention, for the preparation of a medicament for the prophylaxis and/or the treatment of a disease in a mammal, said disease being characterized by obesity and/or insulin resistance and/or glucose intolerance and/or hyperglycemia.

A sixth aspect of the present invention relates to a method of treatment and/or prophylaxis of a disease in a mammal, said disease being characterized by obesity and/or insulin resistance and/or glucose intolerance and/or hyperglycemia, comprising administration of a therapeutically effective amount of the composition according to the present invention.

A seventh aspect of the present invention relates to a method of treatment and/or prophylaxis of a disease in a mammal, said disease being characterized by obesity and/or insulin resistance and/or glucose intolerance and/or hyperglycemia, comprising administration of a therapeutically effective amount of the lipid component according to the present invention.

### DESCRIPTION OF THE FIGURES

FIG. 1

Body weight gain after 9 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R).

* significant difference vs. cHF-fed mice.

FIG. 2

Wet weight of intra-abdominal (gonadal) white adipose tissue after 9 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R).

* significant difference vs. cHF-fed mice.

FIG. 3

Wet weight of subcutaneous (dorsolumbar) white adipose tissue after 9 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R).

* significant difference vs. cHF-fed mice.

FIG. 4

The concentration of total triacylglycerols (TAG) in plasma after 9 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R).

* significant difference vs. cHF-fed mice;

† significant difference vs. G3+R mice.

FIG. 5

Blood glucose concentrations assessed in overnight-fasted (15-hr) mice after 8 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R).

* significant difference vs. cHF-fed mice;

† significant difference vs. G3+R mice.

FIG. 6

Glycemic curves in mice subjected to intraperitoneal (i.p.) glucose tolerance test after 8 weeks of treatment. Blood glucose concentrations were assessed before and 15, 30, 60, 120, and 180 min after the injection of D-glucose (1 mg/g body weight). Mice were fed a composite high-fat diet (cHF; lipid content ˜35.4% wt/wt) or cHF-based diet supplemented with the combination of a phospholipid concentrate G3 and rosiglitazone (G3+R).

*, significant difference between cHF and G3+R mice.

FIG. 7

Glucose tolerance expressed as total area under the glycemic curve (AUC) in mice subjected to glucose tolerance test after 8 weeks of treatment. Mice were fed a composite high-fat diet (cHF; lipid content ˜35.4% wt/wt) or cHF-based diets supplemented with either a phospholipid concentrate G3 alone (G3), rosiglitazone (R), or both (G3+R). *, significant difference vs. cHF-fed mice.

FIG. 8

Change in body weight gain after 8 weeks of treatment. Mice were fed a composite high-fat diet (cHF; lipid content ˜35.4% wt/wt) with or without metformin (2 g/kg diet) or cHF-based diets supplemented with metformin and either a phospholipid concentrate G3 alone (G3), pioglitazone (P), or both (G3+P).

# significant difference before and after the treatment.

* significant difference vs. mice fed the cHF diet containing metformin.

FIG. 9

Change in glucose tolerance following 8 weeks of treatment. Glucose tolerance was expressed as total area under the glycemic curve (total AUCglucose) in mice subjected to glucose tolerance testing before and after 8 weeks of treatment. Mice were fed a composite high-fat diet (cHF; lipid content ˜35.4% wt/wt) with or without metformin (2 g/kg diet) or cHF-based diets supplemented with metformin and either a phospholipid concentrate G3 alone (G3), pioglitazone (P), or both (G3+P).

# significant difference before and after the treatment.

FIG. 10

Change in fasting blood glucose (FBG) concentrations assessed in overnight-fasted (15-hr) mice subjected to glucose tolerance testing before and after 8 weeks of treatment. Mice were fed a composite high-fat diet (cHF; lipid content ˜35.4% wt/wt) with or without metformin (2 g/kg diet) or cHF-based diets supplemented with metformin and either a phospholipid concentrate G3 alone (G3), pioglitazone (P), or both (G3+P).

* significant difference vs. mice fed the cHF diet containing metformin.

# significant difference before and after the treatment.

FIG. 11

The concentration of total triacylglycerols (TG) in plasma after 9 weeks of treatment. Mice were fed a composite high-fat diet (cHF; lipid content ˜35.4% wt/wt) with or without metformin (2 g/kg diet) or cHF-based diets supplemented with metformin and either a phospholipid concentrate G3 alone (G3), pioglitazone (P), or both (G3+P).

* significant difference vs. mice fed the cHF diet containing metformin.

### DETAILED DESCRIPTION OF THE INVENTION

Although a number of compositions have proven to be effective in reducing and treating insulin resistance, adult onset diabetes and MS and its related complications, there is a continuing need to provide novel compositions having improved effect on the above-mentioned conditions and/or less side effects.

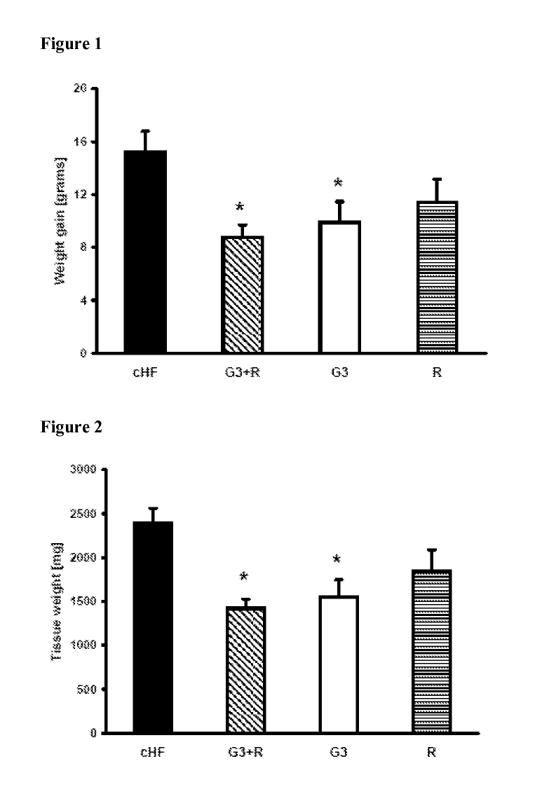

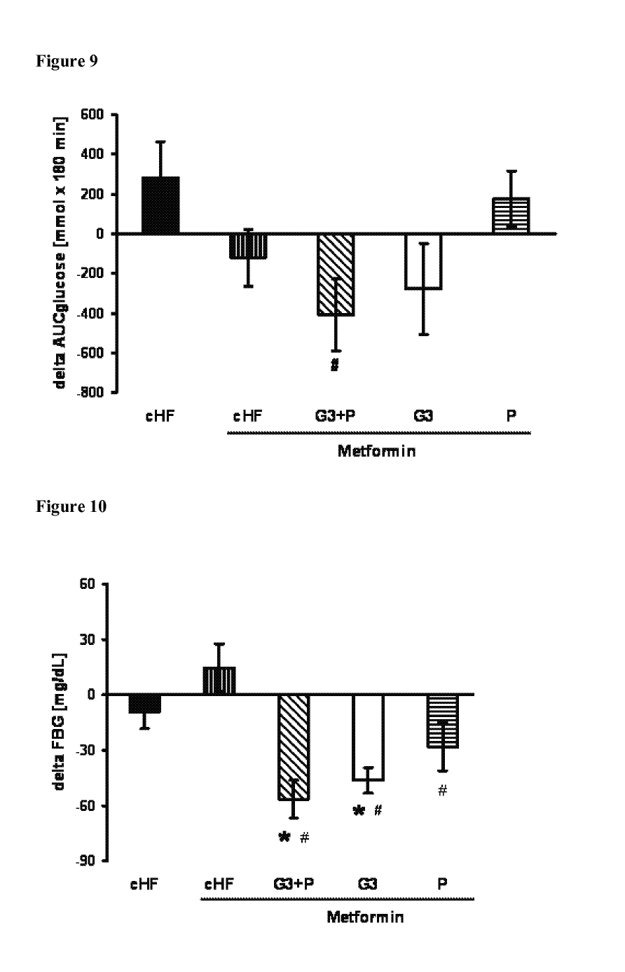

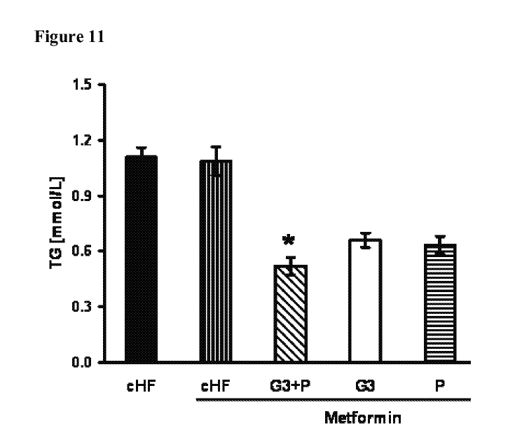

Now, surprisingly in a well established animal model of MS caused by insulin resistance, the obesity prone C57BL/6N mouse, it has now been found that compared with mice fed a high-fat diet, in which the main lipid constituent is corn oil (control), mice fed the same diet supplemented with either a phospholipid concentrate, rosiglitazone, or a combination thereof reduced both body weight (data not shown) and body weight gain (FIG. 1). The effects of the combination treatment tended to be stronger compared with either component alone. Similar results to those obtained when using rosiglitazone have been obtained when using pioglitazone (FIG. 8). Neither the phospholipid concentrate nor rosiglitazone exerted any effect on food consumption.

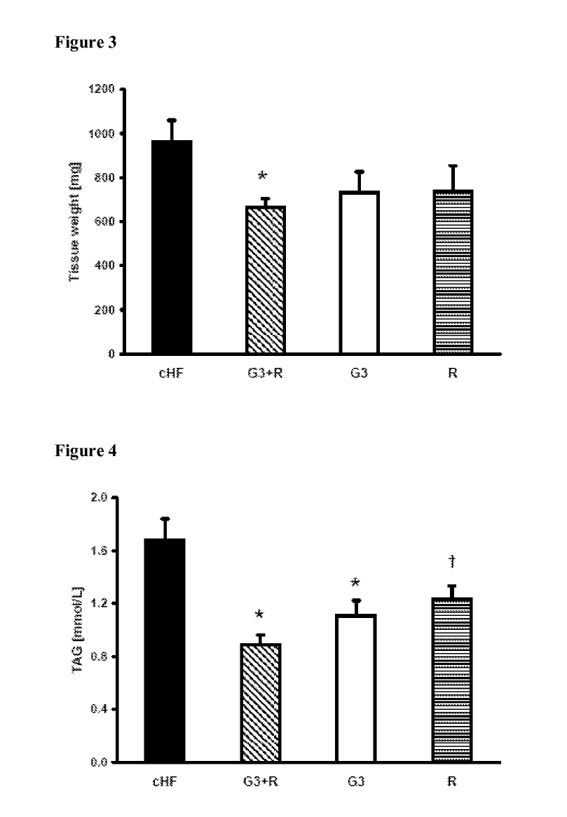

The effects on body weight and body weight gain correlated with the reduction of adiposity, with both gonadal fat in the abdomen (FIG. 2) and subcutaneous adipose tissue (FIG. 3) showing the strongest reduction in weight in the case of the combination treatment.

Further, compared with control, phospholipid concentrate and phospholipid concentrate+rosiglitazone treatments significantly lowered plasma triglyceride levels (FIG. 4), while the effect of the combination treatment tended to be stronger compared with phospholipid concentrate alone, and it was significantly stronger compared with rosiglitazone alone. Similar results to those obtained when using rosiglitazone have been obtained when using pioglitazone (FIG. 11).

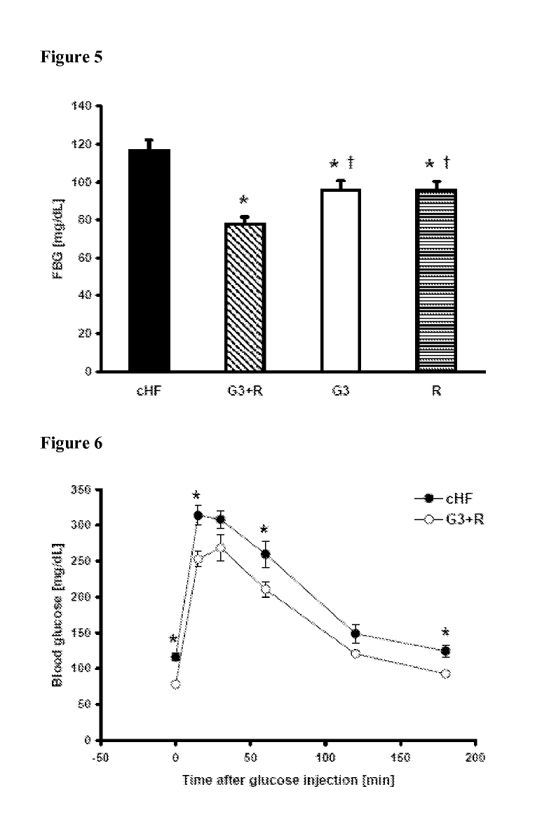

Compared with control (mice fed a composite high-fat diet), all the treatments (phospholipid concentrate, rosiglitazone, and the combination thereof) significantly lowered fasted blood glucose levels (FIG. 5), while the effect of the combination treatment (phospholipid concentrate+rosiglitazone) was significantly stronger compared with phospholipid concentrate or rosiglitazone alone. Similar results to those obtained when using rosiglitazone have been obtained when using pioglitazone (FIG. 10).

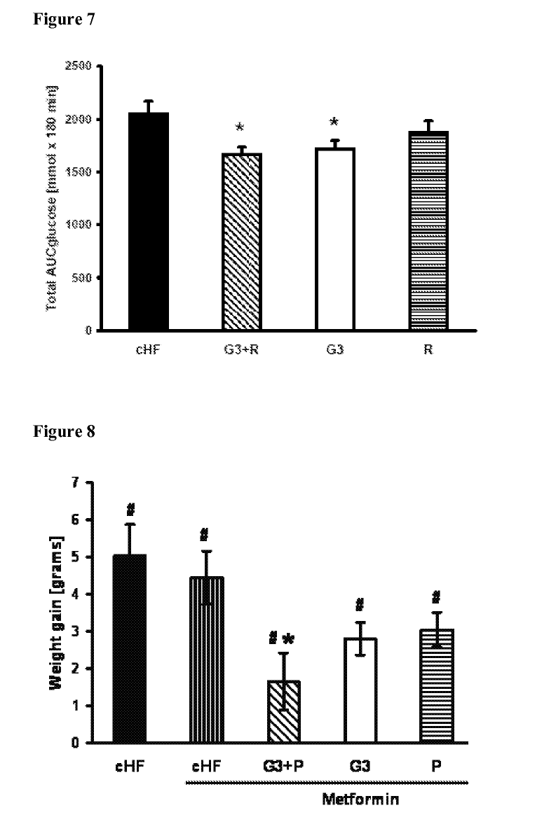

Glucose tolerance assessed by intraperitoneal glucose tolerance test (FIG. 6 and FIG. 7) was significantly improved by phospholipid concentrate and phospholipid concentrate+rosiglitazone treatment, as demonstrated by the suppression of the rise in glycemia following glucose injection (FIG. 6) as well as by the reduction in the area under the glycemic curve (AUC), which was most pronounced in case of the combination phospholipid concentrate+rosiglitazone treatment (FIG. 7). Similar results to those obtained when using rosiglitazone have been obtained when using pioglitazone (FIG. 9).

The above results show that both the phospholipid treatment as well as the phospholipid+rosiglitazone treatment and phospholipid+pioglitazone treatment exerted unexpectedly strong beneficial effects on metabolic disease associated disorders of glucose and lipid metabolism as well as obesity in mice.

Thus, a first aspect of the present invention relates to a composition, comprising at least one peroxisome proliferator-activated receptor (PPAR) agonist and a lipid component, wherein at least 30% (w/w) of said lipid component is phospholipids.

One embodiment according to the first aspect of the present invention relates to a composition comprising at least one PPAR agonist and a lipid component, wherein at least 30% (w/w) of said lipid component is phospholipids; with the proviso that said lipid component is different from the at least one PPAR agonist.

By “PPAR agonist” is meant any compound that increases the biological activity or expression of one or more PPARs (e.g., PPARα, PPARγ, and PPARβ/δ) in a cell. Examples of PPARγ agonists include any of the thiazolidinediones, but particularly rosiglitazone (e.g. Avandia™, GlaxoSmithKline), troglitazone, and pioglitazone (e.g. Actos™, Takeda Pharmaceuticals), and analogs thereof. Rosiglitazone is the preferred PPAR agonist. Additional examples of PPARγ agonists include non-steroidal anti-inflammatory drugs, such as indomethacin, ibuprofen, naprosyn, and fenoprofen, and antioxidants such as vitamin E, vitamin C, S-adenosyl methionine, selenium, beta-carotene, idebenone, cysteine, dithioerythritol, dithionite, dithiothreitol, and pyrosulfite. Examples of PPARα agonists include any of the fibrates (e.g., fenofibrate, bezafibrate, gemfibrozil, and analogs thereof), docosahexaenoic acid, and Wy 4643.

The lipid component according to the first aspect of the present invention is preferably derived from a marine organism such as e.g. fish. More preferably said lipid component is derived from fish meal such as e.g. herring meal.

As described above, the composition according to the present invention comprises the two active components referred to as a PPAR agonist and a lipid component. It is preferred that said lipid component constitutes at least 10% (w/w) of said composition, more preferably at least 20% (w/w) of said composition, even more preferably at least 30% (w/w), such as at least 40% (w/w) or at least 50% (w/w) of said composition, and most preferably at least 70% (w/w) of said composition, e.g. at least 80% (w/w) of said composition or at least 90% (w/w) of said composition.

The above mentioned lipid component is rich in phospholipids. Preferably, at least 30% (w/w), such as at least 40% (w/w), of said lipid component is phospholipids, more preferably at least 60% (w/w), such as e.g. at least 65% (w/w), even more preferably at least 70% (w/w), such as e.g. at least 75% (w/w), and most preferably at least 80% (w/w), such as e.g. at least 85% (w/w).

In one embodiment according to the present invention, at least 50% (w/w) of said phospholipids is phosphatidylcholine (PC), preferably at least 60% (w/w) such as e.g. at least 70% (w/w) or at least 80% (w/w). Further, it is preferred that at least 5% (w/w) of said phospholipids are phosphatidylethanolamine (PE), such as e.g. at least 10% (w/w), at least 20% (w/w), at least 30% (w/w) or at least 40% (w/w). In one preferred embodiment said phospholipids comprise 50-80% (w/w) PC and 5-40% (w/w) PE, preferably 60-80% (w/w) PC and 15-40% (w/w) PE. In another embodiment, the PC:PE ratio (w/w) is in the range 8:1 to 2:1, preferably in the range 5:1 to 2:1 such as e.g. 4:1 to 2:1.

In yet another embodiment according to the present invention, not more than 8% (w/w) of said phospholipids is phosphatidylinositol (PI), preferably not more than 4% (w/w), more preferably not more than 2% (w/w). It is preferred that not more than 8% (w/w) of said phospholipids is phosphatidylserine (PS), preferably not more than 4% (w/w), more preferably not more than 2% (w/w). Further, it is also preferred that not more than 8% (w/w), such as not more than 4% (w/w), of said phospholipids is sphingolipin (SPH).

In one preferred embodiment, said lipid component is the product obtainable by the process described in example 1.

A second aspect of the present invention relates to the composition according to the first aspect of the present invention for use as a medicament.

A third aspect of the present invention relates to a pharmaceutical composition comprising a pharmaceutically effective amount of the composition according to the first aspect of the present invention and a pharmaceutically acceptable carrier.

By “pharmaceutically effective amount” is meant an amount sufficient to provide medical benefit.

Pharmaceuticals/medicaments of this invention may be formulated as pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical formulations of a therapeutically effective amount of a compound of the invention, or pharmaceutically acceptable salt-thereof, is preferably administered orally in an admixture with a pharmaceutically acceptable carrier adapted for the route of administration.

Liquid or solid dosage forms for oral administration include pharmaceutically acceptable emulsions, solutions, suspensions, syrups, soft gelatin capsules, and tablets. These forms contain inert diluents commonly used in the art, such as water or an oil medium. Besides such inert diluents, compositions can also include adjuvants, such as wetting agents, emulsifying agents, and suspending agents.

A fourth aspect of the present invention relates to use of the composition according to the first aspect of the present invention, for the preparation of a medicament for the prophylaxis and/or the treatment of a disease in a mammal, said disease being characterized by insulin resistance and/or glucose intolerance and/or hyperglycemia.

A fifth aspect of the present invention relates to use of the lipid component as defined in the first aspect of the present invention, for the preparation of a medicament for the prophylaxis and/or the treatment of a disease in a mammal, said disease being characterized by insulin resistance and/or glucose intolerance and/or hyperglycemia.

In a preferred embodiments, said disease is selected from the group consisting of diabetes, obesity and hypertriglyceridemia. In a particularly preferred embodiment said disease is diabetes, in particular type 2 diabetes.

In one preferred embodiment according to the fourth and fifth aspect of the present invention, the medicament is administered orally, optionally in an admixture with a pharmaceutically acceptable carrier adapted for the route of administration. Suitable liquid or solid dosage forms for oral administration are mentioned above.

In one preferred embodiment according to the fourth aspect of the present invention, said PPAR agonist is administered in a dosage of from 0.01 mg/kg/day to 0.7 mg/kg/day, preferably in a dosage of from 0.01 mg/kg/day to 0.5 mg/kg/day, more preferably in a dosage of from 0.01 mg/kg/day to 0.3 mg/kg/day.

If said composition comprises rosiglitazone, it is preferred that rosiglitazone is administered in a dosage of from 0.015 mg/kg/day to 0.1 mg/kg/day, such as e.g. a dosage of from 0.02 mg/kg/day to 0.1 mg/kg/day, a dosage of from 0.02 mg/kg/day to 0.8 mg/kg/day or a dosage of from 0.05 mg/kg/day to 0.6 mg/kg/day.

If said composition comprises pioglitazone, it is preferred that pioglitazone is administered in a dosage of from 0.1 mg/kg/day to 0.7 mg/kg/day, such as e.g. a dosage of from 0.2 mg/kg/day to 0.7 mg/kg/day, a dosage of from 0.5 mg/kg/day to 0.7 mg/kg/day or a dosage of from 0.5 mg/kg/day to 0.6 mg/kg/day.

In one preferred embodiment according to the fourth and fifth aspect of the present invention, said lipid component is administered in a dosage of from 80 mg/kg/day to 2 mg/kg/day, preferably in a dosage of from 70 mg/kg/day to 3 mg/kg/day, more preferably in a dosage of from 60 mg/kg/day to 3.5 mg/kg/day.

A sixth aspect of the present invention relates to a method of treatment and/or prophylaxis of a disease in a mammal, said disease being characterized by obesity and/or insulin resistance and/or glucose intolerance and/or hyperglycemia, comprising administration of a therapeutically effective amount of the composition according to the first aspect of the present invention.

A seventh aspect of the present invention relates to a method of treatment and/or prophylaxis of a disease in a mammal, said disease being characterized by obesity and/or insulin resistance and/or glucose intolerance and/or hyperglycemia, comprising administration of a therapeutically effective amount of the lipid component according to the present invention.

In a preferred embodiments, said disease is selected from the group consisting of diabetes, obesity and hypertriglyceridemia. In a particularly preferred embodiment said disease is diabetes, in particular type 2 diabetes.

In one preferred embodiment according to the sixth and seventh aspect of the present invention, the composition according to the first aspect of the present invention or the lipid component as defined in the first aspect of the present invention is administered orally, optionally in an admixture with a pharmaceutically acceptable carrier adapted for the route of administration. Suitable liquid or solid dosage forms for oral administration are mentioned above.

In one preferred embodiment according to the sixth and seventh aspect of the present invention, said lipid component is administered in a dosage of from 80 mg/kg/day to 2 mg/kg/day, preferably in a dosage of from 70 mg/kg/day to 3 mg/kg/day, more preferably in a dosage of from 60 mg/kg/day to 3.5 mg/kg/day.

In one preferred embodiment according to the sixth aspect of the present invention, said PPAR agonist is administered in a dosage of from 0.01 mg/kg/day to 0.7 mg/kg/day, preferably in a dosage of from 0.01 mg/kg/day to 0.5 mg/kg/day, more preferably in a dosage of from 0.01 mg/kg/day to 0.3 mg/kg/day.

In one embodiment according to the seventh aspect of the present invention, said lipid component is administered in a dosage of from 80 mg/kg/day to 2 mg/kg/day.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the scope of the appended claims.

### EXAMPLES

### Example 1

### Preparation of Phospholipid Concentrate (G3)

Extraction of a phospholipid preparation was done according to the method described by Hjaltason, Haraldsson, and Halldorsson (U.S. Pat. No. 7,063,855). Fishmeal from herring was used as starting material and extraction was initiated by addition of ethanol as solvent. The mixture was stirred at room temperature for 3 hours, a temperature where the triglycerides in the fishmeal do not precipitate. This was followed by cooling of the lipid/solvent mixture to a temperature where a significant portion of the triglycerides precipitates. The precipitate was then removed by filtration and the solvent phase was distilled in vacuum on a rotary evaporator to obtain a crude phospholipid-rich component. This phospholipid rich product reached concentrations between 70 and 80% (w/w), the residue being about 4% (w/w) free fatty acids (20% (w/w) docosahexaenoic acid, DHA and 8% (w/w) eicosahexaenoic acid, EPA) and 20-30% (w/w) triglycerides. The phospholipid fraction contained the following components:

### Phosphatidylcholine (PC): 75.9%

### Phosphatidylethanolamine (PE): 18.0%

### Phosphatidylinositol (PI): 1.6%

### Phosphatidylserine (PS): 1.4%

### Sphingolipin (SPH): 3.1%

Total yield of phospholip.: 76%

### Example 2

### In Vivo Experiments

### Rosiglitazone+G3

Experiments were performed on male C57BL/6N mice (Charles River Laboratories, Sulzfeld, Germany). Mice (2 animals per cage) were maintained in a controlled environment (22° C.; 12-h light-dark cycle; light from 6.00 a.m.) and fed ad libitum a standard laboratory chow (lipid content ˜3.4% wt/wt; extruded Ssniff R/M-H diet; Ssniff Spezialdieten GmbH, Soest, Germany). Starting at 3 months of age, animals were randomly divided into 4 groups (n=8) and fed various high-fat diets for subsequent 9 weeks (see table 1). A high-fat control group was maintained on a high-fat diet [cHF; in which corn oil was the main lipid constituent; lipid content ˜35.2% wt/wt; the diet was prepared as described in “Ruzickova J. Rossmeisl M, Prazak T, et al. Omega-3 PUFA of marine origin limit diet-induced obesity in mice by reducing cellularity of adipose tissue; lipids 2004; 39:1177-1185”; see also Table 1], while remaining groups were assigned to the following ‘treatments’ by cHF-based diets supplemented with: (i) both phospholipid concentrate (prepared in example 1) and rosiglitazone (G3+R); (ii) phospholipid concentrate (prepared in example 1) alone (G3); and (ii) rosiglitazone alone (R); see Table 1.

TABLE 1
Diets and treatments

| | Control diet | Treatments | | |
|---|---|---|---|---|
| | cHF | G3 | R | G3 + R |
| Macronutrient composition | | | | |
| Lipids (% diet, wt/wt) | 35.2 | 35.2 | 35.2 | 35.2 |
| Carbohydrates (% diet, | 35.4 | 35.4 | 35.4 | 35.4 |
| wt/wt) | | | | |
| Proteins (% diet, wt/wt) | 20.5 | 20.5 | 20.5 | 20.5 |
| Energy density (kJ/g) | 22.8 | 22.8 | 22.8 | 22.8 |
| Supplement | | | | |
| Phospholipid concentrate | 0 | 32 | 0 | 32 |
| G3 (%)* | | | | |
| Rosiglitazone (mg/kg diet)^{\} | 0 | 0 | 10 | 10 |

*Percentage of dietary lipids replaced by G3 concentrate
^{\}Rosiglitazone (e.g. Avandia(TM), GlaxoSmithKline) was admixed to cHF diet.

Fresh ration of food was distributed daily. Food consumption (24-hr period) and body weights were recorded once a week (body weight gain after 9 weeks of treatment, see FIG. 1). Food intake of a group of mice in each cage was determined by weighing the rest of food remaining in a given cage after a preweighed amount of fresh food had been given (9.00-11.00 a.m.).

Total triacylglycerol in plasma after 9 weeks of treatment (FIG. 4) were determined as previously described (see Ruzickova J, Rossmeisl M, Prazak T, et al. Omega-3 PUFA of marine origin limit diet-induced obesity in mice by reducing cellularity of adipose tissue. Lipids 2004; 39:1177-1185).

An intraperitoneal glucose tolerance test was performed in overnight (−15-hr) fasted animals before and after 8 weeks of dietary treatments. Tail blood was sampled prior to and at 15, 30, 60, 120, and 180 min after i.p. injection of D-glucose (1 mg/g body weight) and blood glucose was assessed by the use of calibrated glucometers (LifeScan, Milpitas, Calif.) (FIG. 5-6). Glucose tolerance was expressed as the total area under the glucose curve (total AUC glucose) (FIG. 7).

After 9 weeks of treatment, mice were killed by cervical dislocation. Intra-abdominal (gonadal) (FIG. 2) and subcutaneous dorsolumbar (FIG. 3) white adipose tissue were quantitatively dissected.

Statistical significance was evaluated by One Way ANOVA followed by the Tukey test for all pairwise multiple comparisons. All values are presented as means±SEM. Comparisons were judged to be significant at p≦0.05.

### Example 3

### In Vivo Experiments

### Pioglitazone+Metformin+G3

Experiments were performed on male C57BL/6J mice (animal facility of the Institute of Physiology, Prague, Czech Republic). Mice (2-4 animals per cage) were maintained in a controlled environment (22° C.; 12-h light-dark cycle; light from 6.00 a.m.) and fed ad libitum a standard laboratory chow (STD; lipid content ˜3.4% wt/wt; extruded Ssniff R/M-H diet; Ssniff Spezialdieten GmbH, Soest, Germany). At the age of 3 months, all animals were assigned to a high-fat diet (cHF; corn oil as the main lipid constituent; lipid content ˜35.2% wt/wt; the diet was prepared as described in “Ruzickova J, Rossmeisl M, Prazak T, et al. Omega-3 PUFA of marine origin limit diet-induced obesity in mice by reducing cellularity of adipose tissue; lipids 2004; 39:1177-1185”; see also Table 1) and fed for a period of 4 months to induce obesity. Subsequently, mice were divided into 5 groups (n=8-9; 2-4 mice/cage) and fed various high-fat diets for the following 9 weeks (see table 2).

TABLE 2
Diets and treatments

| | Control diets | | Treatments | | |
|---|---|---|---|---|---|
| | cHF | cHF + M | G3 | P | G3 + P |
| Macronutrient composition | | | | | |
| Lipids (% diet, wt/wt) | 35.2 | 35.2 | 35.2 | 35.2 | 35.2 |
| Carbohydrates (% diet, wt/wt) | 35.4 | 35.4 | 35.4 | 35.4 | 35.4 |
| Proteins (% diet, wt/wt) | 20.5 | 20.5 | 20.5 | 20.5 | 20.5 |
| Energy density (kJ/g) | 22.8 | 22.8 | 22.8 | 22.8 | 22.8 |
| Supplement | | | | | |
| Metformin (g/kg diet)^{a} | 0 | 2 | 2 | 2 | 2 |
| Phospholipid concentrate G3 | 0 | 0 | 32 | 0 | 32 |
| (%)^{b} | | | | | |
| EPAX 1050 TG (%)^{c} | 0 | 0 | 0 | 0 | 0 |
| Pioglitazone (mg/kg diet)^{d} | 0 | 0 | 0 | 50 | 50 |

^{a}Metforminum Hydrochloride (SIOFOR 850; Berlin - Chemie A. G.) was admixed to cHF diet.
^{b}Percentage of dietary lipids replaced by G3 concentrate;
^{c}Percentage of dietary lipids replaced by EPAX 1050 TG concentrate (containing 50% DHA and 10% EPA; EPAX AS, Lysaker, Norway);
^{d}Pioglitazone Hydrochloride (Actos; Takeda Pharmaceutical) was admixed to cHF diet. cHF + M, cHF diet supplemented with metformin.

Fresh ration of food was distributed daily. Food consumption and body weights were recorded once a week. Food intake of a group of mice in each cage was determined by weighing the rest of food remaining in a given cage after a preweighed amount of fresh food had been given (9.00-11.00 a.m.).

Body weight gain was assessed 2 weeks after the initiation of experimental feeding (period of adaptation to experimental diet) and after 8 weeks of treatment (FIG. 8).

Total triacylglycerol in plasma after 9 weeks of treatment (FIG. 11) were determined as previously described (see Ruzickova J, Rossmeisl M, Prazak T, et al. Omega-3 PUFA of marine origin limit diet-induced obesity in mice by reducing cellularity of adipose tissue. Lipids 2004; 39:1177-1185).

An intraperitoneal glucose tolerance test was performed in animals before and after 8 weeks of dietary treatments. Tail blood was sampled prior to and at 15, 30, 60, 120, and 180 min after i.p. injection of D-glucose (1 mg/g body weight) and blood glucose was assessed by the use of calibrated glucometers (LifeScan, Milpitas, Calif.). The effects of the treatments on glucose tolerance was calculated as a change in glucose tolerance (expressed as area under the glucose curve; AUCglucose), assessed before and after the treatment in each mouse (FIG. 9).

Change in fasting blood glucose (FBG) concentrations was assessed in overnight-fasted (15-hr) mice subjected to glucose tolerance testing before and after 8 weeks of treatment (FIG. 10).

After 9 weeks of treatment, mice were killed by cervical dislocation.

Statistical significance was evaluated by One Way ANOVA followed by the Tukey test for all pairwise multiple comparisons. All values are presented as means±SEM. Comparisons were judged to be significant at p≦0.05.

1. Composition, comprising at least one peroxisome proliferator-activated receptor (PPAR) agonist and a lipid component, wherein at least 30% (w/w) of said lipid component is phospholipids.

2. Composition according to claim 1, wherein at least 50% (w/w) of said lipid component is phospholipids.

3. Composition according to claim 1, wherein at least 70% (w/w) of said lipid component is phospholipids.

4. Composition according to claim 1, wherein said PPAR agonist is a thiazolidinedione selected from the group consisting of rosiglitazone, pioglitazone and troglitazone.

5. Composition according to claim 4, wherein said thiazolidinedione is rosiglitazone and/or pioglitazone.

6. Composition according to claim 1, wherein said lipid component is derived from a marine organism.

7. Composition according to claim 1, wherein said lipid component is derived from fish meal.

8. Composition according to claim 7, wherein said fish meal is herring meal.

9. Composition according to claim 1, wherein said lipid component constitutes at least 50% (w/w) of said composition.

10. Composition according to claim 1, wherein at least 60% (w/w) of said phospholipids is phosphatidylcholine (PC).

11. Composition according to claim 1, wherein at least 10% (w/w) of said phospholipids are phosphatidylethanolamine (PE).

12. Composition according to claim 1, wherein said phospholipids comprise 60-80% (w/w) PC and 10-20% (w/w) PE.

13. Composition according to claim 1, wherein the PC:PE ratio (w/w) is in the range 8:1 to 2:1.

14. Composition according to claim 1, for use as a medicament.

15. Pharmaceutical composition comprising a pharmaceutically effective amount of the composition according to claim 1 and a pharmaceutically acceptable carrier.

16. Method of treatment and/or prophylaxis of a disease in a mammal, said disease being characterized by obesity and/or insulin resistance and/or glucose intolerance and/or hyperglycemia, comprising administration of a therapeutically effective amount of the composition according to claim 1.

17. Method of treatment and/or prophylaxis of a disease in a mammal, said disease being characterized by obesity and/or insulin resistance and/or glucose intolerance and/or hyperglycemia, comprising administration of a therapeutically effective amount of the lipid component as defined in claim 1.

18. Method according to claim 16, wherein said disease is selected from the group consisting of diabetes, obesity and hypertriglyceridemia.

19. Method according to claim 18, wherein said disease is diabetes.

20. Method according to claim 19, wherein said disease is type 2 diabetes.

21. Method according to 16, wherein said administration is oral administration.

22. Method according to claim 16, wherein said PPAR agonist is administered in a dosage of from 0.01 mg/kg/day to 0.7 mg/kg/day and said lipid component is administered in a dosage of from 80 mg/kg/day to 2 mg/kg/day.

23. Method according to claim 17, wherein said lipid component is administered in a dosage of from 80 mg/kg/day to 2 mg/kg/day.
